(19) United States
(12) Patent Application Publication
(10) Pub. No.: US 2026/0345672 A1
(43) Pub. Date: Nov. 5, 2026

(54) Localized Delivery of Sodium Thiosulfate Nanoparticles to Mitigate Arterial Calcification

(71) Applicant: UNIVERSITY OF PITTSBURGH - OF THE COMMONWEALTH SYSTEM OF HIGHER EDUCATION, PITTSBURGH, PA (US)

(72) Inventors: David A. Vorp, Pittsburgh, PA (US); Justin Sol Weinbaum, Allison Park, PA (US); Brittany Rodriguez, Pittsburgh, PA (US); Peter Wipf, Pittsburgh, PA (US)

(21) Appl. No.: 19/124,313

(22) PCT Filed: Oct. 27, 2023
(86) PCT No.: PCT/US23/78080

§ 371 Date: Apr. 25, 2025

Related U.S. Application Data
(60) Provisional application No. 63/381,420, filed on Oct. 28, 2022.

(51) Int. Cl. A61K 33/04; A61K 9/51; A61K 47/64; A61P 9/10

(52) U.S. Cl. CPC A61K 33/04; A61K 9/5169; A61K 47/6435; A61P 9/10

(57) ABSTRACT

Provided herein is a method of mitigating, reducing, or treating cardiovascular calcification, treating atherosclerosis, solubilizing vascular calcium, and/or softening calcium in blood vessels of a patient, including administering an amount of sodium thiosulfate-containing nanoparticles to a blood vessel or blood of the patient effective to mitigate, reduce, or treat cardiovascular calcification, treat atherosclerosis, solubilize vascular calcium, and/or soften calcium in blood vessels of the patient.

### CROSS REFERENCE TO RELATED APPLICATIONS

The present application claims the benefit of U.S. Provisional Patent Application No. 63/381,420, filed Oct. 28, 2022, the content of which is incorporated herein by reference in its entirety.

### BACKGROUND OF THE INVENTION

### Field of the Invention

Provided herein are compositions and methods for treating cardiovascular conditions, and, in particular non-limiting embodiments, compositions including nanoparticles for treating arterial calcification.

### Description of Related Art

Cardiovascular disease is the leading cause of death worldwide, causing about 18 million deaths worldwide in 2019. A root pathology of cardiovascular disease is atherosclerosis, which is defined as the hardening and narrowing of arteries due to calcium and cholesterol accumulation into a plaque. Many studies and treatments have focused on stabilizing and shrinking plaques using cholesterol-lowering drugs. However, studies have shown that around ⅓ of patients have coronary arterial calcification (CAC), and on average 20% of plaque makeup is composed of calcification (including the highly insoluble form of calcium, calcium phosphate (CaP)) within atherosclerotic coronary arteries. In addition, correlations have led scientists to believe that calcification leaves a plaque more prone to rupture due to localized mechanical stresses in the tissue surrounding the calcium deposits. Therefore, the prevalence and risks associated with calcification within the plaque raises concerns in providing treatments to remove calcification from the plaque effectively and safely.

Sodium thiosulfate (STS), an FDA-approved drug to treat calciphylaxis, interacts with CaP to make calcium thiosulfate, which is more soluble in water than CaP and can therefore be more easily removed from tissue. Recent in vitro studies have shown a significant reduction of calcium using STS. However, clinical and in vivo studies have shown negative side effects, including hallucinations, pain in the joints, ringing in the ears, and the like. These side effects show that systemically administering STS intravenously through the body is not a viable options. Accordingly, there is a need in the art for improved delivery systems for STS and for treating cardiovascular disease.

### SUMMARY OF THE INVENTION

Provided herein is a method of mitigating, reducing, or treating cardiovascular calcification, treating atherosclerosis, solubilizing vascular calcium, and/or softening calcium in blood vessels of a patient, including administering an amount of sodium thiosulfate-containing nanoparticles to a blood vessel or blood of the patient effective to mitigate, reduce, or treat cardiovascular calcification, treat atherosclerosis, solubilize vascular calcium, and/or soften calcium in blood vessels of the patient.

Also provided herein is a method of mitigating, reducing or treating cardiovascular calcification, treating atherosclerosis, solubilizing vascular calcium, and/or softening calcium in a patient's blood vessel, including: administering one or more sodium thiosulfate-containing nanoparticles to the patient; and administering a statin to the patient, thereby treating the atherosclerosis, solubilizing the vascular calcium, and/or or softening the calcium in the patient's blood vessels.

Also provided herein is a dosage form including a composition comprising one or more sodium thiosulfate-containing nanoparticles

Additional non-limiting embodiments are set forth in the following numbered clauses:

Clause 1. A method of mitigating, reducing, or treating cardiovascular calcification, treating atherosclerosis, solubilizing vascular calcium, and/or softening calcium in blood vessels of a patient, comprising administering an amount of sodium thiosulfate-containing nanoparticles to a blood vessel or blood of the patient effective to mitigate, reduce, or treat cardiovascular calcification, treat atherosclerosis, solubilize vascular calcium, and/or soften calcium in blood vessels of the patient.

Clause 2. The method of clause 1, wherein the sodium thiosulfate-containing nanoparticles are administered to an atherosclerotic plaque of the patient.

Clause 3. The method of clause 1 or clause 2, wherein the cardiovascular calcification is associated with an atherosclerotic plaque in the patient.

Clause 4. The method of any of clauses 1-3, wherein the patient is a human patient.

Clause 5. The method of any of clauses 1-4, wherein the nanoparticles are administered by inhalation.

Clause 6. The method of any of clauses 1-5, wherein the nanoparticles are administered systemically.

Clause 7. The method of any of clauses 1-6, wherein the nanoparticles are administered parenterally.

Clause 8. The method of any of clauses 1-7, wherein the nanoparticles are administered intravenously.

Clause 9. The method of any of clauses 1-8, wherein the nanoparticles are administered locally to an atherosclerotic plaque.

Clause 10. The method of any of clauses 1-9, wherein the nanoparticles are administered with a balloon catheter.

Clause 11. The method of any of clauses 1-10, wherein the nanoparticles comprise liposomes.

Clause 12. The method of any of clauses 1-11, wherein the nanoparticles comprise lysosomes.

Clause 13. The method of any of clauses 1-12, wherein the nanoparticles comprise

a crown ether.

Clause 14. The method of any of clauses 1-13, wherein the nanoparticles comprise

a nanocarrier.

Clause 15. The method of any of clauses 1-14, wherein the nanocarrier comprises a cyclodextrin and/or a rotaxane.

Clause 16. The method of any of clauses 1-15, wherein the nanoparticles comprise iron nanoparticles.

Clause 17. The method of any of clauses 1-16, wherein the nanoparticles are administered in conjunction with one or more additional atherosclerosis treatments.

Clause 18. The method of any of clauses 1-17, wherein the one or more additional atherosclerosis treatments comprise a statin.

Clause 19. The method of any of clauses 1-18, wherein the nanoparticles are conjugated to a cell adhesion molecule (CAM).

Clause 20. The method of any of clauses 1-19, wherein the nanoparticles are conjugated to a collagen.

Clause 21. The method of any of clauses 1-19, wherein the nanoparticles are conjugated to collagen type IV.

Clause 22. A method of mitigating, reducing or treating cardiovascular calcification, treating atherosclerosis, solubilizing vascular calcium, and/or softening calcium in a patient's blood vessel, comprising: administering one or more sodium thiosulfate-containing nanoparticles to the patient; and administering a statin to the patient, thereby treating the atherosclerosis, solubilizing the vascular calcium, and/or or softening the calcium in the patient's blood vessels.

Clause 23. A dosage form comprising a composition comprising one or more sodium thiosulfate-containing nanoparticles.

Clause 24. The dosage form of clause 23, wherein the nanoparticles comprise liposomes.

Clause 25. The dosage form of clause 23 or clause 24, wherein the nanoparticles comprise lysosomes.

Clause 26. The dosage form of any of clauses 23-25, wherein the nanoparticles comprise a crown ether.

Clause 27. The dosage form of any of clauses 23-26, wherein the nanoparticles comprise a nanocarrier.

Clause 28. The dosage form of any of clauses 23-27, wherein the nanocarrier comprises a cyclodextrin and/or a rotaxane.

Clause 29. The dosage form of any of clauses 23-28, wherein the nanoparticles comprise iron nanoparticles.

Clause 30. The dosage form of any of clauses 23-29, wherein the nanoparticles are conjugated to a cell-adhesion molecule (CAM).

Clause 31. The dosage form of any of clauses 23-30, wherein the nanoparticles are conjugated to a collagen.

Clause 32. The dosage form of any of clauses 23-31, wherein the nanoparticles are conjugated to collagen type IV.

Clause 33. The dosage form of any of clauses 23-32, wherein the nanoparticles are conjugated to a collagen-binding peptide.

Clause 34. The dosage form of any of clauses 23-33, wherein the nanoparticles are conjugated to a collagen type IV binding peptide.

Clause 35. The dosage form of any of clauses 23-34, wherein the nanoparticles are formulated for inhalation.

Clause 36. The dosage form of any of clauses 23-35, wherein the nanoparticles are formulated for systemic administration.

Clause 37. The dosage form of any of clauses 23-36, wherein the nanoparticles are formulated for parenteral administration.

Clause 38. The dosage form of any of clauses 23-37, wherein the nanoparticles are formulated for intravenous administration.

Clause 39. Use of the dosage form of any of clauses 23-38 for treatment of atherosclerosis.

Clause 40. Use of the dosage form of any of clauses 23-38 for treatment of endothelial dysfunction.

Clause 41. Use of the dosage form of any of clauses 23-38 for treatment of coronary artery disease.

### BRIEF DESCRIPTION OF THE DRAWING

FIG. 1 depicts an overall reduction of calcium phosphate within a collagen gel model after being treated with 1-10 mM of STS for a week; and

FIG. 2 depicts a microCT scan of a collagen gel as shown in FIG. 1.

### DESCRIPTION OF THE INVENTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with ‘including’, ‘containing’, or ‘characterized by’. The term “consisting essentially of” limits the scope of a claim to the specified materials or steps and those that do not materially affect basic and novel characteristic(s). The term “consisting of” excludes any element, step, or ingredient not specified in the claim. As used herein, embodiments “comprising” one or more stated elements or steps also include but are not limited to embodiments “consisting essentially of” and “consisting of” these stated elements or steps.

As used herein, the term “patient” or “subject” refers to members of the animal kingdom including but not limited to human beings and “mammal” refers to all mammals, including, but not limited to human beings.

Provided herein are compositions of sodium thiosulfate (STS), and dosage forms of such compositions, useful for methods of treating cardiovascular conditions. In non-limiting embodiments, STS is provided in a systemically injectable nanoparticle treatment that can target/localize to an atherosclerotic plaque within a vessel of a patient and remove calcium deposits from that plaque, or from other areas where calcium deposits may be found within the vasculature or other area of the body. In non-limiting embodiments the nanoparticles are of a size <100 nm) encapsulating sodium thiosulfate and conjugated with a collagen binding peptide. Such delivery provides for localized STS treatment that can increase the solubility and removal of calcium.

Suitable compositions and dosage forms useful in the methods described herein include STS. STS is FDA approved for treating calcium deposits within the skin (e.g., calciphylaxis), and STS-containing medicaments are disclosed in U.S. Pat. No. 11,142,456 and European Patent Application Publication No. 2451435, the contents of which are incorporated herein by reference in their entirety. Without wishing to be bound by the theory, it is believe that STS reacts with the calcium to create calcium thiosulfate, thus making calcium, for example calcium within the arterial wall, more soluble and degradable by the body.

In terms of in vivo calcium for which STS is useful, calcium phosphate is water insoluble and is normally found within diseased arteries. In comparison, calcium thiosulfate is water soluble. Therefore, the conversion from, for example, calcium phosphate to calcium thiosulfate, increases the solubility, and is expected to facilitate the degradation of the insoluble calcium. Treatment with STS for calcium deposits within arteries through systemic injections have been attempted, but the side-effect profile is unacceptable. Accordingly, the methods described herein are not expected to be accompanied by any significant side effects (e.g., pain, tinnitus, hallucinations) and are not expected to be accompanied by any of the significant side effects attributable to systemic free-STS administration.

In non-limiting embodiments, methods of administering STS as described herein target exposed vascular basement membrane of any atherosclerotic plaque within the body. In non-limiting embodiments, administration of nanoparticles containing STS allow for deeper penetration of the STS to the intimal and/or medial layers of the vessel, for example through fusion of the nanoparticles with cells in the various layers of the vessel and delivery of STS in that manner.

In terms of administration to, and treatment of calcium deposits in the medial layer of a blood vessel, calcium in that area may compromise the compliance and pulsatility flow of the vessel in question. Thus, in non-limiting embodiments, efficacy of administration of compositions containing STS-containing nanoparticles may be confirmed by compression tests, used to measure any differences in the mechanical properties of the vessel when decreasing the calcium within the surrounding walls. Without wishing to be bound by the theory, it is believed that the expected outcome of this treatment will reduce the circumferential stress in the vessel (e.g., an arterial wall) by about 10%, about 20%, about 30%, about 40%, about 50%, and/or greater, all values and subranges therebetween inclusive.

In non-limiting embodiments, administration of compositions containing STS nanoparticles decreases the amount of calcium within the fibrous cap, and decreases a plaque's vulnerability to rupture. As used herein, the term “fibrous caps” refers to an accumulation of smooth muscle cells that accumulate beneath a plaque endothelium. The fibrous cap is a protective layer of connective tissue surrounding the plaque. Studies have shown that peak circumferential stress increases ~2-fold when microcalcifications lie within the fibrous cap, which as noted above may render the atherosclerotic plaque more vulnerable to rupture. Coronary arterial calcification (CAC) scores derived from CT scans may be used to predict the risk of heart attacks or strokes and most consider a fibrous cap without microcalcifications as more stable. Thus, in non-limiting embodiments, administration of STS-containing nanoparticles lowers the patient's CAC score. Those of skill in the art will appreciate that a CAC score may be based on the Agatston Scale, which is understood to refer to total area of calcium deposits and the density of the calcium. In the Agatston Scale, 0=no calcium deposits (e.g., no risk of heart disease), 1-10=low levels of deposits and a low risk (less than 10%) of heart disease, 11-99=mild calcium deposits, 100-399=moderate calcium deposits, 400-999=severe calcium deposits, and 1000+=extreme deposits, with a 25% risk of a heart attack within a year. In non-limiting embodiments, the patient has an Agatston Scale score of 1-10, 11-99, 100-399, or 400-999, all values and subranges therebetween inclusive, and, following treatment with the STS-containing nanoparticles as described herein, has an Agatston Scale score of 0, 1-10, 11-99, or 100-399, all values and subranges therebetween inclusive.

In addition to degradation of calcium and/or plaques, administration of compositions containing STS-containing nanoparticles may be employed to reverse endothelial dysfunction, for example in patients with calciphylaxis. As used herein, the term “endothelial dysfunction” refers to a non-obstructive vascular disease, such as coronary artery disease (CAD), in which there are no blockages, but the vessel is nonetheless narrowed.

In terms of administration of compositions containing STS-containing nanoparticles, any suitable dosage regimen will fall within the scope of the present disclosure. In non-limiting embodiments, a therapeutically-effective amount of STS is delivered through nanoparticles as described herein. A “therapeutically-effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. In non-limiting embodiments, a prophylactically-effective amount of STS is delivered through nanoparticles as described herein. A “prophylactically-effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). Exemplary dosage regimens are described herein. However, in non-limiting embodiments, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., and may be proportionally reduced or increased as indicated by the exigencies of the therapeutic situation. In some instances, it may be especially advantageous to formulate compositions in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

In non-limiting embodiments, administration of STS-containing nanoparticles occurs more than once, for example, twice, three times, four times, five times, or more, all values and subranges inclusive. Administration of STS-containing nanoparticles may occur over minutes, hours, days, weeks, months, and/or years all values and subranges therebetween inclusive. In non-limiting embodiments, STS-containing nanoparticles are administered every two weeks. Without wishing to be bound by the theory, it is believed that such a dosing schedule allows for collagen remodeling. In non-limiting embodiments, treatment is continued until there is a visible reduction in calcification (e.g., CAC score is reduced, as described herein).

In non-limiting embodiments, compositions containing STS-containing nanoparticles are administered concurrently with a standard-of-care atherosclerosis treatments, including, without limitation, diets high in fruits and vegetables, grains, and/or low in saturated fat, sodium, and/or added sugars, exercise, reduction and/or cessation of smoking, reduction and/or cessation of alcohol consumption, increased sleep quality and/or duration, and/or pharmacological interventions. Suitable pharmacological interventions are known to those of skill in the art and may include, without limitation, angiotensin-converting enzyme (ACE) inhibitors, beta blockers, anti-platelet medications, anti-clotting medications, calcium channel blockers, medications that control blood sugar (such as empagliflozin, canagliflozin, and liraglutide), metformin, nitrate (such as nitroglycerin), ranolazine, statins, other cholesterol-lowering medications (including, for example, ezetimibe, PCSK9 inhibitor, bempedoic acid, and omega-3 fatty acids), and/or thrombolytic medications.

### Delivery of STS

As described herein, for the disclosed methods STS is delivered in nanoparticles. Useful dosage forms for STS-containing nanoparticles include, for example and without limitation: parenteral, intravenous, intramuscular, intraocular, and/or intraperitoneal solutions, oral tablets and/or liquids, topical drops, formulations for inhalation, ointments, creams, and transdermal devices (e.g., patches). The compositions including STS-containing nanoparticles may include a sterile solution comprising the nanoparticles and a solvent, such as water, saline, lactated Ringer's solution, or phosphate-buffered saline (PBS). Additional excipients, such as polyethylene glycol, emulsifiers, salts and buffers may be included in the solution. In non-limiting embodiments, STS as described herein is provided in nanoparticles, for example in liposomes, for administration parenterally, by injection (e.g., intravenously), and/or by inhalation. Suitable dosage forms may include single-dose or multiple-dose vials or other containers, medical syringes (e.g., pre-filled syringes), droppers (e.g., eye droppers), containers for inhalation devices, inhalation devices, and the like. In non-limiting embodiments, compositions containing STS-containing nanoparticles may be delivered through a coated stent and/or a coated balloon catheter, directly to a site of a plaque in a vessel, for example as shown (with other active agents) in Tzafriri et al., Balloon-based drug coating delivery to the artery wall is dictated by coating micro-morphology and angioplasty pressure gradients, Biomaterials 2020, 260:120337.

Therapeutic compositions, including those containing STS, may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to administration. Such dosage forms can be prepared according to conventional methods known to those skilled in the art of pharmaceutical science, including those described herein.

The pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein may also comprise liposomes, micelles, microspheres, nanosystems, and mixtures thereof.

The pharmaceutical compositions provided herein may be micronized to a size suitable for delivery by inhalation, such as about 50 micrometers or less, about 10 micrometers or less and/or about 100 nm or less, all values and subranges therebetween inclusive. Particles of such sizes may be prepared using a comminuting method known to those skilled in the art, such as spiral jet milling, fluid bed jet milling, supercritical fluid processing to form nanoparticles, high pressure homogenization, or spray drying.

Pharmaceutical formulations adapted for oral, nasal, and/or parenteral administration (including intravenous administration) include aqueous and non-aqueous sterile solutions which may contain, in addition to the active pharmaceutical ingredient or drug, for example and without limitation, anti-oxidants, buffers, bacteriostats, lipids, liposomes, lipid nanoparticles, emulsifiers, suspending agents, and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous solutions and suspensions may be prepared from sterile powders, granules and tablets.

Pharmaceutical formulations adapted for nasal administration wherein the carrier is a solid include a coarse powder having a particle size, for example, in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include, without limitation, fine particle dusts or mists which may be generated by means of various types of metered dose pressurized aerosols, nebulizers or insufflators. In the context of delivery of the active agents described herein by inhalation, inhalation drug products, such as metered-dose inhalers, as are broadly-known in the pharmaceutical arts, are used. Metered dose inhalers are configured to deliver a single dose of an active agent per actuation, though multiple actuations may be needed to effectively treat a given patient.

Pharmaceutical formulations adapted for parenteral administration include aqueous and non-aqueous sterile injection solutions which may contain, for example and without limitation, anti-oxidants, buffers, bacteriostats, lipids, liposomes, emulsifiers, also suspending agents and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets.

Therapeutic/pharmaceutical formulations as described herein may be prepared in accordance with acceptable pharmaceutical procedures, such as described in Remington: The Science and Practice of Pharmacy, 21^{st} edition, ed. Paul Beringer et al., Lippincott, Williams & Wilkins, Baltimore, MD Easton, Pa. (2005) (see, e.g., Chapters 37, 39, 41, 42 and 45 for examples of powder, liquid, parenteral, intravenous and oral solid formulations and methods of making such formulations).

Therapeutic compositions typically must be sterile and stable under the conditions of manufacture and storage. For example, sterile injectable solutions can be prepared by incorporating the active agent in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Pharmaceutically acceptable salts of the compounds, such as STS, described herein also may be used in the methods described herein. Pharmaceutically acceptable salt forms of the compounds described herein may be prepared by conventional methods known in the pharmaceutical arts, and include as a class veterinarily-acceptable salts. For example and without limitation, where a compound comprises a carboxylic acid group, a suitable salt thereof may be formed by reacting the compound with an appropriate base to provide the corresponding base addition salt. Non-limiting examples include: alkali metal hydroxides, such as potassium hydroxide, sodium hydroxide and lithium hydroxide; alkaline earth metal hydroxides, such as barium hydroxide and calcium hydroxide; alkali metal alkoxides, such as potassium ethanolate and sodium propanolate; and various organic bases such as piperidine, diethanolamine, and N-methylglutamine.

Non-limiting examples of pharmaceutically-acceptable base salts include: aluminum, ammonium, calcium, copper, ferric, ferrous, lithium, magnesium, manganic, manganous, potassium, sodium, and zinc salts. Salts derived from pharmaceutically acceptable organic non-toxic bases include, without limitation: salts of primary, secondary, and tertiary amines, substituted amines including naturally occurring substituted amines, cyclic amines, and basic ion exchange resins, such as arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

Non-limiting examples of pharmaceutically-acceptable acid salts include: acetate, adipate, alginate, arginate, aspartate, benzoate, besylate (benzenesulfonate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate, galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, and phthalate.

Multiple salts forms are also considered to be pharmaceutically-acceptable salts. Common, non-limiting examples of multiple salt forms include: bitartrate, diacetate, difumarate, dimeglumine, diphosphate, disodium, and trihydrochloride.

As such, “pharmaceutically acceptable salt” as used herein is intended to mean an active ingredient (drug) comprising a salt form of any compound as described herein. The salt form may confer improved and/or desirable pharmacokinetic/pharmodynamic properties of the compounds described herein.

### STS-Containing Nanoparticles

As described herein, STS is incorporated in, or coated on, nanoparticles, nanocarriers, crown ethers, and/or liposomes, for delivery. In non-limiting embodiments, the delivery is lysosome-mediated delivery. In this disclosure, the terms “nanoparticle”, “nanocarrier”, and “liposome” are used interchangeably. Those of skill in the art are familiar with methods of forming nanoparticles and/or liposomes, for example as set forth in Xiaojiao Yu, et al. “Design of Nanoparticle-Based Carriers for Targeted Drug Delivery,” Journal of Nanomaterials, vol. 2016, Article ID 1087250, 15 pages, 2016, Rajesh Singh, et al., “Nanoparticle-based targeted drug delivery” Exp Mol Pathol. 2009 June; 86(3): 215-223, and Xuan Ding et al., “Extended Release and Targeted Drug Delivery Systems”, in Remington The Science and Practice of Pharmacy, David B. Troy Ed. 2006 Lippincott Williams & Wilkins, Chapter 47). In non-limiting embodiments, the term “liposome” refers to a vesicle composed of amphiphilic lipids arranged in at least one bilayer, e.g., one bilayer or a plurality of bilayers. In non-limiting embodiments, liposomes as described herein include phosphatidylcholine, and/or derivatives thereof.

With regard to lysosomal delivery, as used herein the term refers to use of nanoparticles designed to either actively bind to surface receptors of a cell, allowing for receptor-mediated internalization (e.g. via phagocytosis or clathrin-mediated endocytosis) or to enter cells passively via diffusion (including macropinocytosis). Such systems are known to those of skill in the art, for example as set forth in Sun et al., Lysosomal-mediated drug release and activation for cancer therapy and immunotherapy, Adv. Drug Deliv. Rev. 2023, 192:114624 and Sharma et al., Lysosomal targeting strategies for design and delivery of bioactive for therapeutic interventions, J Drug Target. 2018, 26(3): 208-221. Other suitable nanocarriers for lysosomal delivery of STS include cyclodextrin and/or polyrotaxane-based carriers. In non-limiting embodiments, the nanocarrier is a beta-cyclodextrin, for example a cyclodextrein-threaded biocleavable polyrotaxane, for example as disclosed in Tamura et al., Beta-cyclodextrin-threaded biocleavable polyrotaxanes ameliorate impaired autophagic flux in Niemann-Pick Type C disease, J. Biol. Chem. 2015, 290(15): 9442-9454. In non-limiting embodiments the nanocarrier is 2-hydroxyethoxy)ethyl group-modified biocleavable polyrotaxane bearing terminal disulfide linkages (HEE-SS-PRX).

With regard to use of crown ethers, as used herein that term refers to cyclic compounds made up of repeating ether units (R—O—R′), where the crown results in a cavity within the ring, which can be used to load a variety of bioactive compounds. Typically, a crown ether includes at least four oxygen atoms, and the oxygen atoms may each be separated by, typically, two or three carbon atoms. Crown ethers may include any ether, including ethyleneoxy (—CH₂CH₂O—)ₙ, in any number of repeats (e.g., n=4, 6, 8, 10, 12, 14, 16, 18, 20, etc.). Suitable crown ethers include at least 8-crown-4, 12-crown-4, 14-crown-4, 15-crown-5, 18-crown-6, 21-crown-7, etc., up to at least 81-crown-27 (using the naming convention in which the first number refers to the number of atoms in the crown and the second number refers to the number of oxygen atoms), and those of skill in the art will appreciate that the particular crown used depends on the cargo being delivered, as the increased number of atoms in the crown results in increased cavity size. Suitable crowns may also include substitutions, for example with alkyl groups, whether in aliphatic or cyclic form, such as benzene rings (e.g., dibenzo-18-crown-6), as is known by those of skill in the art. In non-limiting embodiments, one or more oxygen atoms within the ether units is replaced with a nitrogen atom, which allows formation of aza crown ethers, and/or with a sulfur atom, which allows for formation of thia-crown ethers. Exemplary structures of crown ethers are provided below:

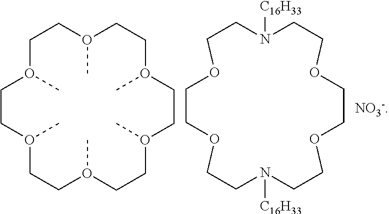

Crown ethers, and their role in drug delivery, are known to those of skill in the art, for example as set forth in Chehardoli et al., The role of crown ethers in drug delivery, Supramolecular Chem. 2019, 4:221-238.

As noted above, in on-limiting embodiments STS is coated on a nanoparticle, for example an inorganic nanoparticle, as is known in the art. In non-limiting embodiments, the inorganic nanoparticle is an iron oxide nanoparticle, for example a magnetic iron oxide, for example as set forth in Turrina et al., Bare iron oxide nanoparticles as drug delivery carrier for the short cationic peptide lasioglossin, Pharmaceuticals 2021, 14(5): 405.

With regard to liposomes, these may include unilamellar and multilamellar vesicles that have a membrane formed from a lipophilic material and an aqueous interior. In non-limiting embodiments, the aqueous portion contains the STS composition. The lipophilic material isolates the aqueous interior from an aqueous exterior, which typically does not include the STS composition, although in some examples, it may. Liposomes are useful for the transfer and delivery of active ingredients to the site of action. Because the liposomal membrane is structurally similar to biological membranes, when liposomes are applied to a tissue, the liposome may fuse with bilayer of the cellular membranes, which, as described herein, may be useful for delivery of STS to deeper layers of vessels. As the merging of the liposome and cell progresses, the internal aqueous contents that include STS are delivered to sites where STS can specifically bind to a target and can act as described herein.

A liposome containing STS can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The STS preparation is then added to the micelles that include the lipid component. The cationic groups on the lipid interact with STS and condense around the STS to form a liposome. After condensation, the detergent is removed, e.g., by dialysis, to yield a liposomal preparation of STS.

Liposomes typically fall into two broad classes. Cationic liposomes are positively charged liposomes which interact with the negatively charged nucleic acid molecules to form a stable complex. The positively charged liposome binds to a negatively charged surface and, in the presence of an acidic environment, the liposomes are ruptured, releasing their contents.

SLiposomes, which are pH-sensitive or negatively charged, may entrap similarly-charged compounds. In such situations, since both the compound and the lipid are similarly charged, repulsion rather than complex formation occurs. Nevertheless, some compound is entrapped within the aqueous interior of these liposomes. pH sensitive liposomes have been used to deliver various compounds.

One type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Non-ionic liposomal systems have also been examined to determine their utility in the delivery of drugs to various tissue, such as the skin. In non-limiting embodiments, non-ionic liposomal systems include a non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) have been used to deliver, for example, cyclosporin-A into different lays of tissue, including the skin, as disclosed in, for example, as disclosed in Glukhova, Liposome drug delivery system across endothelial plasma membrane: role of distance between endothelial cells and blood flow rate, Molecules 2020, 25 (8): 1875.

The term “liposome” also includes “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M}I, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES).

Various liposomes comprising one or more glycolipids are known in the art. U.S. Pat. No. 4,837,028 and WO 88/04924 (the contents of which are incorporated herein by reference in their entirety) disclose liposomes comprising (1) sphingomyelin and (2) the ganglioside G_{M}I or a galactocerebroside sulfate ester. U.S. Pat. No. 5,543,152 (incorporated herein by reference in its entirety) discloses liposomes comprising sphingomyelin. Liposomes comprising 1,2-sn-dimyristoylphosphatidylcholine are disclosed in WO 97/13499 (incorporated herein by reference in its entirety).

In one aspect, cationic liposomes are used. Cationic liposomes possess the advantage of being able to fuse to the cell membrane. Non-cationic liposomes, although typically not able to fuse as efficiently with the plasma membrane, may taken up by macrophages in vivo and can be used to deliver agents to macrophages.

Further advantages of liposomes include biocompatibility and biodegradability, their ability to incorporate a wide range of water and lipid soluble drugs, and/or protection of encapsulated compounds in their internal compartments from metabolism and degradation. Important considerations in the preparation of liposome formulations include, without limitation, the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that are capable of fusing with negatively charged lipids of cell membranes. Another commercially available cationic lipid, 1,2-bis(oleoyloxy)-3,3-(trimethylammonia) propane (“DOTAP”) (Boehringer Mannheim™, Indianapolis, Indiana) differs from DOTMA in that the oleoyl moieties are linked by ester, rather than ether linkages. (DOTAP) can be used in combination with a phospholipid to form vesicles. Lipofectin™ (Bethesda Research Laboratories, Gaithersburg, Md.) is an effective agent for the delivery of anionic compounds that comprise positively charged DOTMA liposomes which interact spontaneously with negatively charged compounds to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells.

Other cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega™, Madison, Wisconsin) and dipalmitoylphosphatidylethanolamine 5-carboxyspermyl-amide (“DPPES”) (see, e.g., U.S. Pat. No. 5,171,678, incorporated herein by reference in its entirety).

Another cationic lipid conjugate includes derivatization of the lipid with cholesterol (“DC-Choi”) which has been formulated into liposomes in combination with DOPE. Lipopolylysine, made by conjugating polylysine to DOPE, has been reported to be effective for transfection in the presence of serum. For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical™, La Jolla, California) and Lipofectamine™ (DOSPA) (Life Technology™, Inc., Gaithersburg, Maryland). Other cationic lipids suitable for delivery of compounds are described in WO 98/39359 and WO 96/37194 (both incorporated herein by reference in their entirety).

The cationic lipid can be, for example, N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA) 1,-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino) ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aHI-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl) (2-hydroxydodecyl)amino)ethyl) piperazin-1-yl)ethylazanediyl)didodecan-2-ol (Tech Gl), or a mixture thereof.

Liposomal formulations may be particularly suited for administration to blood vessels and plaques, and as such liposomes present several advantages over other formulations. Such advantages include reduced side effects related to high systemic absorption of the administered drug, increased accumulation of the administered drug at the desired target, and the ability to administer STS into the targeted tissue.

Liposomes that include STS can be made highly deformable. Such deformability can enable the liposomes to penetrate through pores that are smaller than the average radius of the liposome. For example, transfersomes are a type of deformable liposomes. Transferosomes can be made by adding surface edge activators, usually surfactants, to a standard liposomal composition. Transfersomes that include STS can be delivered, for example, to blood vessels or subcutaneously by infection in order to deliver STS to keratinocytes in the skin. In addition, due to the lipid properties, these transferosomes can be self-optimizing (adaptive to the shape of pores, e.g., in atherosclerotic plaque tissue or skin), self-repairing, and can frequently reach their targets without fragmenting, and often self-loading.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations.

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

STS can also be provided in micellar formulations. As used herein, the term “micelles” is a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation may be prepared by mixing an aqueous solution of STS, a suitable lipid, and micelle forming compounds. Exemplary micelle-forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the lipid. Micelles will form with substantially any kind of mixing of the ingredients but vigorous mixing to provide smaller size micelles.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

In one aspect, the lipid to drug ratio (mass/mass ratio) (e.g., lipid to STS ratio) may be in the range of from about 1:1 to about 50:1, from about 1:1 to about 25:1, from about 3:1 to about 15:1, from about 4:1 to about 10:1, from about 5:1 to about 9:1, or about 6:1 to about 9:1, all values and subranges therebetween inclusive.

In another aspect, lipid nanoparticles (“lipid particles” or “lipid-containing particles”) can be used to deliver the STS. Lipid nanoparticles, as they are broadly-known, may be particles comprising, without limitation: a helper lipid; cholesterol or a derivative thereof; a PEG-based compound, such as a PEG-containing polymer or a PEGylated fatty acid-containing compound such as a PEG-conjugated lipid; and an ionizable lipid (lipidoid). The lipid-containing particles may be described as lipid nanoparticles or lipid microparticles, depending on their size. The particles may be used to deliver any compatible cargo or active agent, such as STS.

The lipid particles may be prepared using any useful method. These include, but are not limited to, spray drying, single and double emulsion solvent evaporation, solvent extraction, phase separation, and simple and complex coacervation, among other methods. The method of preparing the particles may be the double emulsion process and spray drying. The conditions used in preparing the particles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix. Methods developed for making particles for delivery of encapsulated agents are amply described in the literature. In one example, the lipid-containing particles are prepared by microfluidics (see, e.g., Chen D, et al., Rapid discovery of potent siRNA-containing lipid nanoparticles enabled by controlled microfluidic formulation. J Am Chem Soc. 2012 Apr. 25; 134(16):6948-51 and Cayabyab, C, et al., “mRNA Lipid Nanoparticles: Robust low-volume production for screening high-value nanoparticle materials,” Document ID: mrnaspark-AN-1018, (2018) Precision NanoSystems, Inc., describing methods of making lipid nanoparticles, including suitable ratios for various constituents). Briefly, appropriate amounts of the lipidoid, the helper lipid, the cholesterol or cholesterol derivative, and PEG-based material are mixed in an appropriate solvent, such as 90% ethanol and 10% 10 mM sodium citrate and mixed with an appropriate amount of the cargo, such as STS in 10 mM sodium citrate at a weight ratio of STS to the (lipidoid+cholesterol or cholesterol derivative+ helper lipid+PEG-based material) of, for example and without limitation of 1:2-1000, such as from 1:4 to 1:50, e.g., 1:10. The amount of helper lipid in the lipid particle may range from 10 to 80 mol % of the amounts of total lipids, e.g., lipidoid+cholesterol or cholesterol derivative+ helper lipid+PEG-based material in the lipid particle. The lipid particles may be formed in an automated device (such as a microfluidic device) or by rapid pipetting. Particles may be diluted in a suitable aqueous solvent, such as PBS, and optionally dialyzed against the same or a different aqueous solvent.

The lipid-containing particles comprise cholesterol or a derivative thereof, such as 3β[N—(N′,N′-dimethylaminoethane)-carbamoyl]cholesterol (DC-cholesterol). The lipid-containing particles comprise a PEG (poly(oxyethylene))-based material, such as a PEGylated fatty acid-containing compound or PEG-containing block copolymer, such as a polaxamer. Non-limiting examples of PEG-based materials include: PEG-ceramide, PEG-DMG, PEG-PE, poloxamer, or DSPE carboxy PEG. For instance, in certain embodiments, the PEG-based material is C14 PEG2000 DMG, C15 PEG2000 DMG, C16 PEG2000 DMG, C18 PEG2000 DMG, C14 PEG 2000 ceramide, C15 PEG2000 ceramide, C16 PEG2000 ceramide, C18 PEG2000 ceramide, C14 PEG2000 PE, C15 PEG2000 PE, C16 PEG2000 PE, C18 PEG2000 PE, C14 PEG350 PE, C14 PEG5000 PE, poloxamer F-127, poloxamer F-68, poloxamer L-64, or DSPE carboxy PEG. A lipidoid is a lipid-like molecule. An ionizable lipidoid is a lipidoid that forms an ion in acidic or basic conditions. Non-limiting examples of ionizable lipidoids are provided in U.S. Pat. No. 9,439,968, generally forming lipidoids by conjugate addition of alkyl-acrylates to amines. Examples of lipidoids are described in US Patent Application Publication Nos. US20110256175A1 and US20200109113A1, and U.S. Pat. Nos. 7,939,505B2, 8,802,863B2, 8,969,353B2, 9,139,554B2, and 9,227,917B2, incorporated herein by reference for their description of additional exemplary lipidoid compounds, and uses therefor.

The compound 2,2-Dilinoleyl-4-dimethylaminoethyl-[1,3]-dioxolane can be used to prepare nanoparticles. In one aspect, the nanoparticle includes 40% 2, 2-Dilinoleyl-4-dimethylaminoethyl-[1,3]-dioxolane: 10% DSPC: 40% Cholesterol: 10% PEG-C-DOMG (mole percent) with a particle size of 63.0±20 nm and a 0.027 STS/Lipid Ratio.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-0-dimethyl PE, 18-1-trans PE, 1-stearoyl-2-oleoyl-phosphatidyethanolamine (SOPE), cholesterol; or a mixture thereof. The non-cationic lipid can be from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles can be from 0 mol % to about 20 mol % or about 2 mol % of the total lipid present in the particle.

In some aspects, the nanoparticle further includes cholesterol at, e.g., about 10 mol % to about 60 mol % or about 48 mol % of the total lipid present in the particle.

In one aspect, the lipidoid ND98-4HC1 (MW 1487) (see U.S. Patent Application Publication No. 2009/0023673, incorporated herein by reference in it is entirety), Cholesterol (Sigma-Aldrich™), and PEG-Ceramide C16 (Avanti™ Polar Lipids) can be used to prepare nanoparticles (i.e., LNP01 particles). Stock solutions of each in ethanol can be prepared as follows: ND98, 133 mg/ml; cholesterol, 25 mg/ml, PEG-Ceramide C 16, 100 mg/ml. The ND98, cholesterol, and PEG-Ceramide C16 stock solutions can then be combined in a, e.g., 42:48:10 molar ratio. The combined lipid solution can be mixed with aqueous STS (e.g., in sodium acetate pH 5) such that the final ethanol concentration is about 35-45% and the final sodium acetate concentration is about 100-300 mM. STS-containing nanoparticles may form spontaneously upon mixing.

Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

In non-limiting embodiments, the nanoparticle containing STS may be specifically targeted, for example to a plaque, a calcium-rich deposit, and/or a fibrous cap (e.g., smooth muscle cells. Various targeting moieties that may be conjugated to any of the delivery vehicles (e.g., nanoparticles) described herein are known to those of skill in the art, and may include cell adhesion molecules (CAMs), including molecules specific to atherosclerosis. In non-limiting embodiments, targeting moieties and/or targets may include one or more of vascular cell adhesion molecule-1 (VCAM-1), interleukin-4 (IL-4) receptor, stablins (including stablin-2), CD44, cytokine receptor antagonists (including IL-1 receptor antagonist), low-density lipoprotein (LDL), C-C motif chemokine receptor-2 (CCR2), C-C motif chemokine receptor-5 (CCR5), Lyp-1, Apo A-I mimetics (including 18A, 37 pA, and 4F), Apo E, Integrins (including GPIIb-IIIa), Collagens (including Type I and Type IV) Fibrin, ALX/FPR2, Peptides (including cLABL, binding sequence of fibrinogen (y3), VHPKQR, CREKA, and RGD), Antibodies (including to SAINT-O-Somes, ICAM-1, and Anti-platelet endothelial cell adhesion molecule (PECAM)), cytokines (such as IL-10, including recombinant cytokines such as recombinant IL-10), C-terminal globular domain of adiponectin, Peptidomimetic vitronectin antagonist, PLGA-PEG Polymers, e-selectin, p-selectin, integrins (including αvβ3 Integrin), and molecules that bind to the foregoing. Suitable targeting moieties are also described in, for example, Chung, Targeting and therapeutic peptides in nanomedicine for atherosclerosis. Experimental Biology and Medicine 2016, 241 (9): 891-898, Nakhlband et al., Combating atherosclerosis with targeted nanomedicines: recent advances and future prospective. BioImpacts 2018, 8 (1): 59-75, Li et al., Biomimetic nanoparticles targeting atherosclerosis for diagnosis and therapy, Smart Medicine 2023, 2 (3): E20230015, Prilepskii et al., Nano-article-based approaches towards the treatment of atherosclerosis, Pharmaceutics 2020, 12 (11): 1056, and Li et al., Surface-modified nanotherapeutics targeting atherosclerosis, Biomater. Sci. 2022, 10:5459-5471. In non-limiting embodiments, compositions containing nanoparticles containing STS are conjugated to one or more collagen proteins. In non-limiting embodiments, the nanoparticle is conjugated to collagen type IV. In non-limiting embodiments, the nanoparticle is conjugated to a collagen-binding peptide, for example a collagen type IV-binding peptide. In non-limiting embodiments, the collagen-binding peptide comprises a motif of about 6 to about 10 repeating units of Gly-Xaa-Yaa, where the Xaa may be proline and the Yaa may be hydroxyproline. Additional collagen-binding peptides are known, for example as set forth in Farndale, Collagen-binding proteins: insights from the collagen toolkits. Essays Biochem. 2019, 63 (3): 337-348, Abd-Elgaliel et al., Exploring the structural requirements of collagen-binding peptides, Biopolymers 2013, 100 (2): 167-173, and Boone et al., Designing collagen-binding peptide with enhanced properties using hydropathic free energy predicitons, Appl. Sci. 2023, 13 (5): 3342.

### EXAMPLES

### Example 1

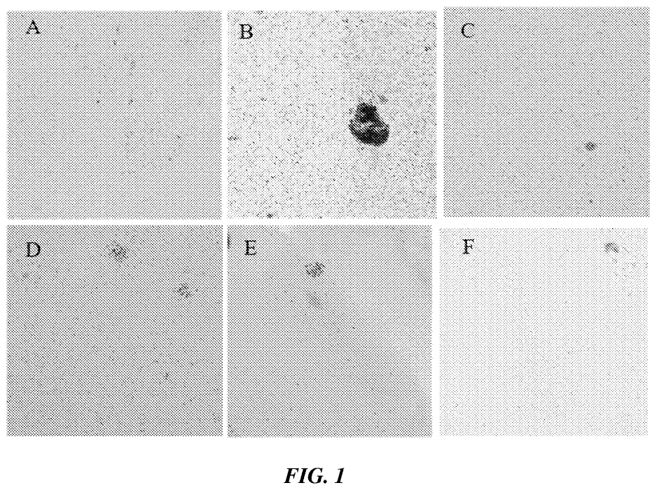

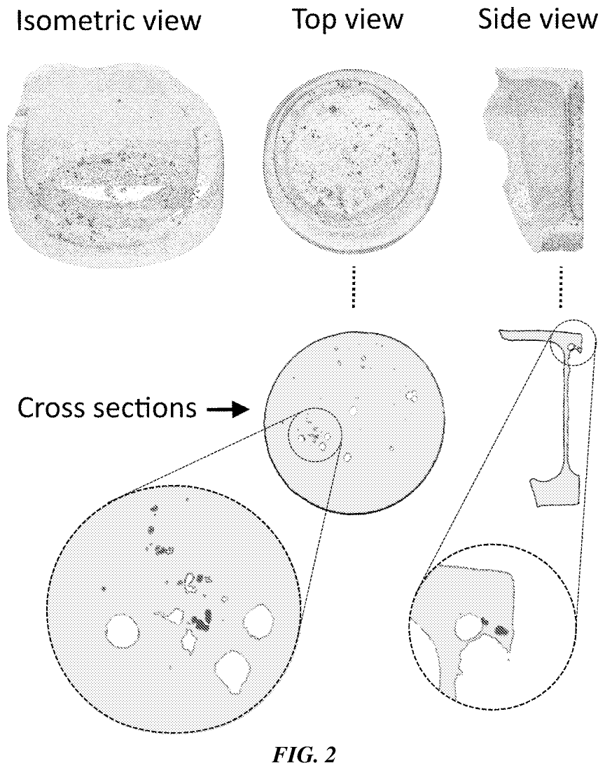

The present study aims to mimic and evaluate the in vitro reaction of STS and CaP within a collagen gel. To test the hypothesis that the insoluble CaP will be removed by the STS, CaP was mixed with a collagen gel solution to mimic the common location of calcium within the artery. STS solution (1 mM, 5 mM, and 10 mM of STS) was placed over the gel and the gel was placed in an incubator. Remaining calcification was analyzed using a Von Kossa stain at 1, 2, 3, 4, 5, 7, or 14 days. Results from the 7-day timepoint are shown in FIGS. 1-2. Specifically, those results show that there is an overall reduction of CaP in the collagen gel after one week of treatment with STS (FIG. 1, panels c-e, 1 mM, 5 mM, and 10 mM, respectively), compared to ultra-pure water (panel b). Panel a shows treatment with EDTA, used as a positive control. Panel f shows a collagen gel without CaP included.

### Example 2

The present study aims to fabricate and validate STS encapsulated nanoparticles conjugated with Col IV (sts-col-IV NPs). This will test the hypothesis that sts-col-IV NPs will encapsulate the targeted amount of STS and accurately bind to collagen IV. Fabrication of the nanoparticles is performed using nanoprecipitation and a double emulation technique using Milli-Q water as the blank group. Once the nanoparticles are fabricated, a release is performed using an Eppendorf rotating wheel and collecting samples at 1, 2, 3, 4, 5, 7, 10, or 14 days. High performance liquid chromatography is used to calculate the amount of STS released from the nanoparticles. In order to measure toxicity to surrounding cells, the nanoparticles are introduced within the media of cultured ECs and SMCs. A live dead stain is used to analyze the results. In order to analyze accurate binding of the nanoparticles, photolithography/PDMS stamp is used to micropattern collagen type IV onto a well plate. sts-col IV NPs are then placed over the micropattern. Light microscopy is used to observe if the NPs targeted the micropatterned areas. If light microscopy cannot accurately visualize the localization of the NPs, GFP/FITC loaded NPs can be fabricated using the same conjugated target to perform this experiment. The goal of this aim is to fabricate, optimize and analyze the success of the NP cargo and targeting method. An alternative approach would be to create STS encapsulated liposomes conjugated with the collagen type IV binding peptide.

### Example 3

The present study aims to evaluate the reaction of sts-col-IV NPs and human atherosclerotic peripheral arteries and any changes in mechanical properties ex vivo. This will test the hypothesis that the sts-col-IV NPs will significantly reduce the calcium within the atherosclerotic tissue and change the mechanical properties of (e.g., soften) the tissue. To analyze the sts-col-IV NP reaction on the calcium within atherosclerotic plaques, 3 mm sections of atherosclerotic arteries with plaque (obtained from lower extremity amputations in diabetics) are treated with the sts-col-IV NPs and blank-col-IV NPs. This experiment is done with different concentrations of STS for 1, 3, 7, and 14 days. The tissue is stained with Von Kossa stain, H&E, and Alizarin Red (immunofluorescence) before and after treatment to quantitate calcification. Opening angle and compression testing is performed to analyze any differences in residual stress, compression stiffness, and max compressive stress before and after the reduction of calcium within the atherosclerotic plaque. The goal of this study is to assess any reduction in calcium using the sts-col-IV NPs and understand the changes in mechanical properties as a result of reduced calcium.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of mitigating, reducing, or treating cardiovascular calcification, treating atherosclerosis, solubilizing vascular calcium, and/or softening calcium in blood vessels of a patient, comprising administering an amount of sodium thiosulfate-containing nanoparticles to a blood vessel or blood of the patient effective to mitigate, reduce, or treat cardiovascular calcification, treat atherosclerosis, solubilize vascular calcium, and/or soften calcium in blood vessels of the patient.

2. The method of claim 1, wherein the sodium thiosulfate-containing nanoparticles are administered to an atherosclerotic plaque of the patient.

3. The method of claim 1, wherein the cardiovascular calcification is associated with an atherosclerotic plaque in the patient.

4. (canceled)

5. The method of claim 1, wherein the nanoparticles are administered by inhalation

6-7. (canceled)

8. The method of claim 1, wherein the nanoparticles are administered intravenously.

9. (canceled)

10. The method of claim 1, wherein the nanoparticles are administered with a balloon catheter.

11. The method of claim 1, wherein the nanoparticles comprise liposomes or lysosomes.

12. (canceled)

13. The method of claim 1, wherein the nanoparticles comprise a crown ether.

14. The method of claim 1, wherein the nanoparticles comprise a nanocarrier.

15. (canceled)

16. The method of claim 1, wherein the nanoparticles comprise iron nanoparticles.

17. The method of claim 1, wherein the nanoparticles are administered in conjunction with one or more additional atherosclerosis treatments.

18. (canceled)

19. The method of claim 1, wherein the nanoparticles are conjugated to a cell adhesion molecule (CAM), a collagen, or a collagen binding peptide.

20-21. (canceled)

22. A method of mitigating, reducing or treating cardiovascular calcification, treating atherosclerosis, solubilizing vascular calcium, and/or softening calcium in a patient's blood vessel, comprising:
administering one or more sodium thiosulfate-containing nanoparticles to the patient; and
administering a statin to the patient,
thereby treating the atherosclerosis, solubilizing the vascular calcium, and/or or softening the calcium in the patient's blood vessels.

23. A dosage form comprising a composition comprising one or more sodium thiosulfate-containing nanoparticles.

24. The dosage form of claim 23, wherein the nanoparticles comprise liposomes or lysosomes.

25. (canceled)

26. The dosage form of claim 23, wherein the nanoparticles comprise a crown ether.

27. The dosage form of claim 23, wherein the nanoparticles comprise a nanocarrier.

28. (canceled)

29. The dosage form of claim 23, wherein the nanoparticles comprise iron nanoparticles.

30. The dosage form of claim 23, wherein the nanoparticles are conjugated to a cell-adhesion molecule (CAM), a collagen, or a collagen-binding peptide.

31-34. (canceled)

35. The dosage form of claim 23, wherein the nanoparticles are formulated for inhalation.

36-37. (canceled)

38. The dosage form of claim 23, wherein the nanoparticles are formulated for intravenous administration.

39-41. (canceled)
